(19) United States
(12) Patent Application Publication
(10) Pub. No.: US 2014/0194713 A1
(43) Pub. Date: Jul. 10, 2014

(54) CONTACT LENSES HAVING TWO-ELECTRODE ELECTROCHEMICAL SENSORS

(71) Applicant: GOOGLE INC., Mountain View, CA (US)

(72) Inventor: Zenghe Liu, Alameda, CA (US)

(73) Assignee: GOOGLE INC., Mountain View, CA (US)

(21) Appl. No.: 14/029,281

(22) Filed: Sep. 17, 2013

Related U.S. Application Data
(63) Continuation of application No. 13/568,078, filed on Aug. 6, 2012.

(51) Int. Cl. A61B 5/1486; A61B 5/00; A61B 5/145

(52) U.S. Cl. CPC A61B 5/1486; A61B 5/14532; A61B 5/14546; A61B 5/6821; A61B 5/0004
USPC 600/347; 600/345

(57) ABSTRACT

Apparatus, systems and methods employing contact lenses with two-electrode electrochemical sensors are provided. In some aspects, the contact lens includes: a substrate that forms at least part of a body of the contact lens; and a circuit, disposed on or within the substrate, and including a two-electrode electrochemical sensor. The two-electrode electrochemical sensor can include: a working electrode; and a combination reference-counter electrode. The electrochemical sensor can be an amperometric sensor that senses a biological feature of a wearer of the contact lens. The working electrode can generate a signal indicative of the sensed analyte, and the combination reference-counter electrode can pass the signal generated from the working electrode. The signal can be employed to determine the analyte concentration of a solution in contact with the contact lens.

### TECHNICAL FIELD

This disclosure generally relates to contact lenses having two-electrode electrochemical sensors.

### BACKGROUND

Traditionally, electrochemical amperometric sensors are three-electrode mode sensors having a reference electrode, working electrode and a counter electrode. The reference electrode controls the electrochemical potential of the working electrode, the working electrode oxidizes or reduces the analyte to generate a sensor signal, and the counter electrode passes the current signal generated from the working electrode. Silver/silver chloride (Ag/AgCl) is typically employed for the reference electrode. However, to make the reference electrode, multiple extra steps have to be used to deposit the Ag metal layer and subsequently choloridize it electrochemically. Accordingly, apparatus and methods for two-electrode electrochemical sensors are desired.

### BRIEF DESCRIPTION OF THE DRAWINGS

FIG. 1 is an illustration of a block diagram of an exemplary non-limiting system that facilitates contact lenses with two-electrode electrochemical sensors in accordance with aspects described herein.

FIG. 2 is an illustration of an exemplary non-limiting diagram of a circuit for a contact lens with a two-electrode electrochemical sensor in accordance with aspects described herein.

FIGS. 3 and 4 are illustrations of exemplary non-limiting graphs detailing the performance of two-electrode electrochemical sensors employed on a contact lens in accordance with aspects described herein.

FIG. 5 is an illustration of an exemplary non-limiting graph detailing the performance of conventional three-electrode electrochemical sensors.

FIG. 6 is an illustration of an exemplary non-limiting graph detailing a comparison of the two-electrode electrochemical sensor employed on a contact lens in accordance with aspects described herein, and a conventional three-electrode mode electrochemical sensor.

FIG. 7 is an illustration of an exemplary calibration curve for the results shown in FIG. 6 in accordance with aspects described herein.

FIGS. 8 and 9 are illustrations of exemplary non-limiting flow diagrams of methods that facilitate contact lenses with two-electrode electrochemical sensors in accordance with aspects described herein.

FIG. 10 is an illustration of a schematic diagram of an exemplary networked or distributed computing environment with which one or more aspects described herein can be associated.

FIG. 11 is an illustration of a schematic diagram of an exemplary computing environment with which one or more aspects described herein can be associated.

### DETAILED DESCRIPTION

Various aspects are now described with reference to the drawings, wherein like reference numerals are used to refer to like elements throughout. In the following description, for purposes of explanation, numerous specific details are set forth in order to provide a more thorough understanding of one or more aspects. It is be evident, however, that such aspects can be practiced without these specific details. In other instances, structures and devices are shown in block diagram form in order to facilitate describing one or more aspects.

Amperometry is the use of electric current or change in electric current to detect analytes in a solution. Amperometry is performed using amperometric electrochemical sensors with electrodes placed in close proximity to the substance being analyzed. The measurements from the electrode are based on an oxidizing or reducing reaction that occurs when the electrode is in proximity to the substance. Proper potentials applied to the electrodes cause such oxidizing or reducing reactions to occur. The resulting electrical currents can be employed in identifying the analyte in some embodiments.

Amperometric biosensors require additional biosensing elements on the working electrode in order for the current-generating redox reaction to occur. One type of biosensing elements is oxidase enzyme that catalyzes the oxidation of analytes by oxygen. For example, glucose oxidase can be deposited onto the biosensor to enable the sensor to detect glucose. Other analytes such as lactate and urea can also be detected in embodiments employing corresponding oxidases.

A number of different technologies can be employed in producing the above-referenced electrodes. For example, for noble metal thin film electrodes, vacuum deposition including sputtering and evaporation can be employed. These steps can be combined with photolithographic techniques to mask and pattern specific electrodes and connections to the electrodes. For carbon and Ag/AgCl electrodes, screen printing of carbon ink can be employed.

Apparatus, systems and methods disclosed herein relate to contact lenses that include amperometric electrochemical sensors. When the amperometric sensor is employed on the contact lens, numerous problems can arise. For example, since the contact lens is in constant contact with aqueous solution (either the contact lens cleaning solution while not in use or tears while in use), the AgCl material of a reference electrode can slowly dissolve into the solution. As a result, the electrode potential can undesirably shift.

To address these issues, in some aspects, a two-electrode sensor on a contact lens is provided. The contact lens can include: a substrate that forms at least part of a body of the contact lens; and a circuit having a working electrode and a combination reference-counter electrode. The circuit can be disposed on or within the substrate.

The sensor can be a biosensor that senses glucose, protein, triglycerides, urea or lactate levels in the wearer of the contact lens. The working electrode can generate a signal indicative of a sensed analyte and the combination reference-counter electrode can pass the signal generated from the working electrode. In one or more embodiments, the working electrode is composed of platinum and the combination reference-counter electrode is composed of a noble metal. Further, the area of the working electrode can be formed to be smaller than the area of the combination reference-counter electrode. The ratio of the area of combination reference-counter electrode to the working electrode combination reference-counter is between 2 and 10000 in some embodiments.

One or more of the aspects can advantageously facilitate a two-electrode electrochemical sensor that does not require a separate reference electrode.

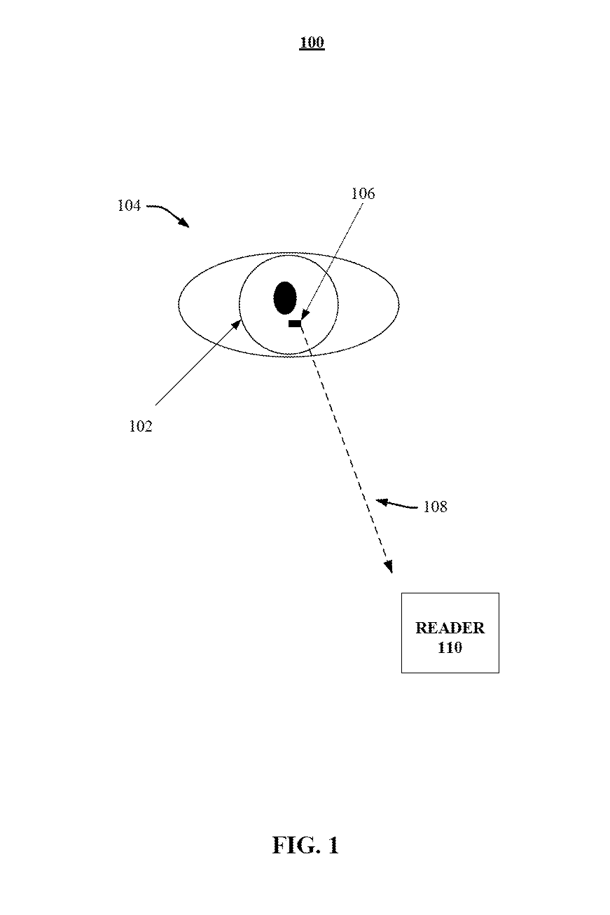

FIG. 1 is an illustration of a block diagram of a system that facilitates contact lenses with two-electrode electrochemical sensors in accordance with aspects described herein. The system 100 includes a contact lens 102 covering at least a portion of an eye 104 and having a circuit 106.

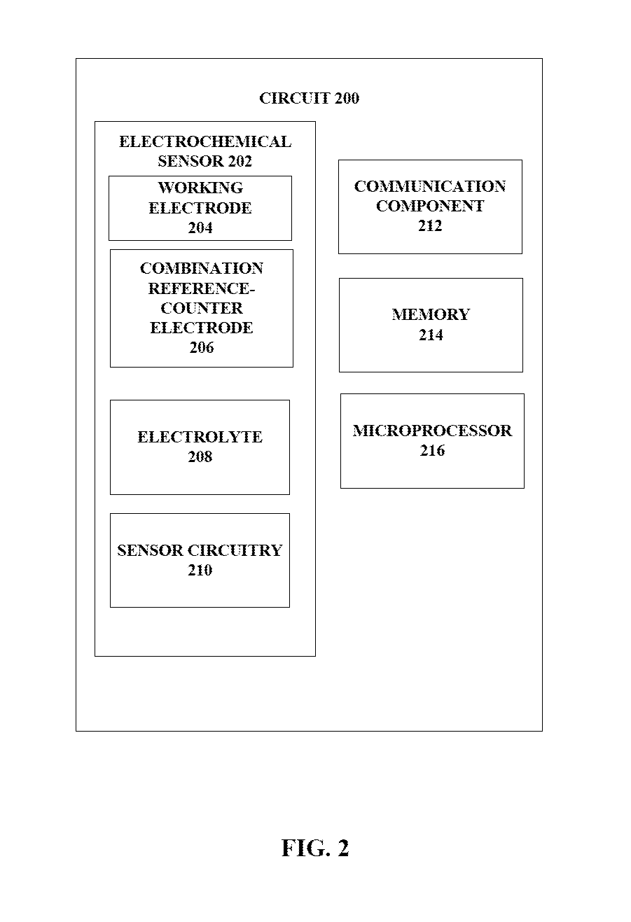

The circuit 106 can be as described in greater detail with reference to FIG. 2. In particular, the circuit 106 can include a two-electrode amperometric electrochemical sensor (not shown).

The two-electrode electrochemical sensor can include a working electrode and a combination reference-counter electrode. The sensor can also include an electrolyte and sensor circuitry. An enzyme (e.g., an oxidase associated with glucose, protein, triglycerides, urea or lactate) can be deposited on the working electrode to enable the electrochemical sensor to sense its substrates. The combination reference-counter electrode can perform the combined functionality of the counter and the reference electrodes of typical three-electrode electrochemical sensors. As such, the combination reference-counter electrode can eliminate the need for a separate reference electrode in the circuit of the contact lens combination reference-counter

Turning back to FIG. 1, the contact lens 102 can detect one or more substances incident on the contact lens 102. For example, in various aspects, the contact lens 102 can detect glucose, protein, triglycerides, urea and/or lactate levels in a body of the wearer of the contact lens 102. The contact lens 102 can perform such detection via the above-described two-electrode amperometric electrochemical sensor of the circuit 106.

In some aspects, the contact lens 102 can include a communication component (not shown) to communicate an analyte concentration of the substance incident on the contact lens 102, an output current generated by the circuit 106 and/or any number of other different types of information. By way of example, but not limitation, the communication component can be or include a radio frequency (RF) antenna. In some aspects, the information 108 can be communicated to a reader 110 external to the contact lens 102. In some aspects, the reader 110 can be an RF reader. Accordingly, the contact lens 102 can have wireless communication with a reader 110.

It is to be appreciated that in accordance with one or more aspects described in this disclosure, users can opt-in or opt-out of providing personal information, demographic information, location information, proprietary information, sensitive information, or the like in connection with data gathering aspects. Moreover, one or more aspects described herein can provide for anonymizing collected, received, or transmitted data.

FIG. 2 is an illustration of an exemplary non-limiting diagram of a circuit for a contact lens with a two-electrode electrochemical sensor in accordance with aspects described herein. In various aspects, the circuit 200 can include one or more of the structure and/or functionality of the circuit 106 (and vice versa).

As shown in FIG. 2, the circuit 200 can include an electrochemical sensor 202, communication component 212, memory 214 and/or microprocessor 216. In some aspects, the circuit 200 can include only the electrochemical sensor 202 and/or only the electrochemical sensor 202 and the communication component 212. One or more of the electrochemical sensor 202, communication component 212, memory 214 and/or microprocessor 216 can be chemically, electrically and/or biochemically coupled to one another to perform one or more functions of the circuit 200.

In various aspects, the circuit 200 can be disposed on or within a substrate of a contact lens (not shown). The circuit 200 can sense an analyte concentration of a solution on or within the contact lens employing the two-electrode electrochemical sensor 202.

The electrochemical sensor 202 can be a two-electrode electrochemical sensor in various aspects. In some aspects, the electrochemical sensor 202 can be an amperometric sensor.

In some aspects, as shown, the electrochemical sensor 202 can include a working electrode 204, combination reference-counter electrode 206, electrolyte 208 and/or sensor circuitry 210. The working electrode 204, combination reference-counter electrode 206, electrolyte 208 and/or sensor circuitry 210 can be electrically, communicatively, chemically and/or biochemically coupled to one another to perform the functions of the electrochemical sensor 202. For example, functions of the electrochemical sensor 202 can include sensing the analyte concentration of a solution incident on the contact lens on which the circuit 200 is disposed.

The working electrode 204 can facilitate determination of the analyte concentration. For example, in some aspects, an enzyme can be deposited on the working electrode 204 of the electrochemical sensor 202 to enable the electrochemical sensor 202 to sense its substrates. For example, in some aspects, glucose oxidase can be deposited on the working electrode 204 of the electrochemical sensor 202. The electrochemical sensor 202 can then become a glucose biosensor. As such, the glucose biosensor can sense a level of glucose in a body of the wearer of the contact lens. In various aspects, the analyte can be glucose, protein, triglycerides, urea, lactate and/or any number of other different types of fluids.

The combination reference-counter electrode 206 can perform the functionality of the counter and the reference electrodes of typical three-electrode electrochemical sensors. As such, the combination reference-counter electrode 206 can eliminate the need for a separate reference electrode in the circuit 200. Accordingly, an Ag/AgCl reference electrode separate from a counter electrode is not required in the aspects described herein.

Accordingly, the working electrode 204 and combination reference-counter electrode 206 can be in contact with the electrolyte 208 in various aspects. Upon sensing of the solution, a chemical reaction can occur and the sensor circuitry 210 can generate an electrical current.

In some aspects, the circuit 200 can include a memory 214 and/or a microprocessor 216. The memory 214 can store information regarding the analyte concentration sensed, output currents and/or computer-executable instructions for execution by the microprocessor 216.

The microprocessor 216 can execute computer-executable instructions to perform one or more functions of the circuit 200. For example, in some aspects, the microprocessor 216 can convert the output current from the electrochemical sensor 202 to an analyte concentration value.

In some aspects, the working electrode 204 and the combination reference-counter electrode 206 can be made of the same material. In some aspects, the two-electrode electrochemical sensor 202 can include a hydrogen peroxide sensor with two Platinum (Pt) electrodes.

However, in some aspects, the working electrode 204 and the combination reference-counter electrode 206 can be made of different materials. In these aspects, for example, the working electrode 204 can be made of Pt and the combination reference-counter electrode 206 can be made of another noble metal (other than Pt). In various aspects, the combination reference-counter electrode 206 can be made of carbon (C).

The design of the two-electrode electrochemical sensor 202 can be such that when a potential (or voltage) is applied across the working electrode 204 and a counter electrode, the current density on the counter electrode can be so low that the potential essentially sits at its open circuit value. As such, a single counter electrode having these characteristics can serve as a combination reference-counter electrode 206. Accordingly, while the component at reference numeral 206 is referred to as a combination reference-counter electrode 206, in various aspects, the combination reference-counter electrode 206 is actually a counter electrode that is much larger than the working electrode 204 such that the current density of the counter electrode is so low that the potential sits at its open circuit value.

By way of example, but not limitation, in some aspects, a ratio of an area of the combination reference-counter electrode 206 and an area of the working electrode 204 is between approximately 2 and approximately 10000.

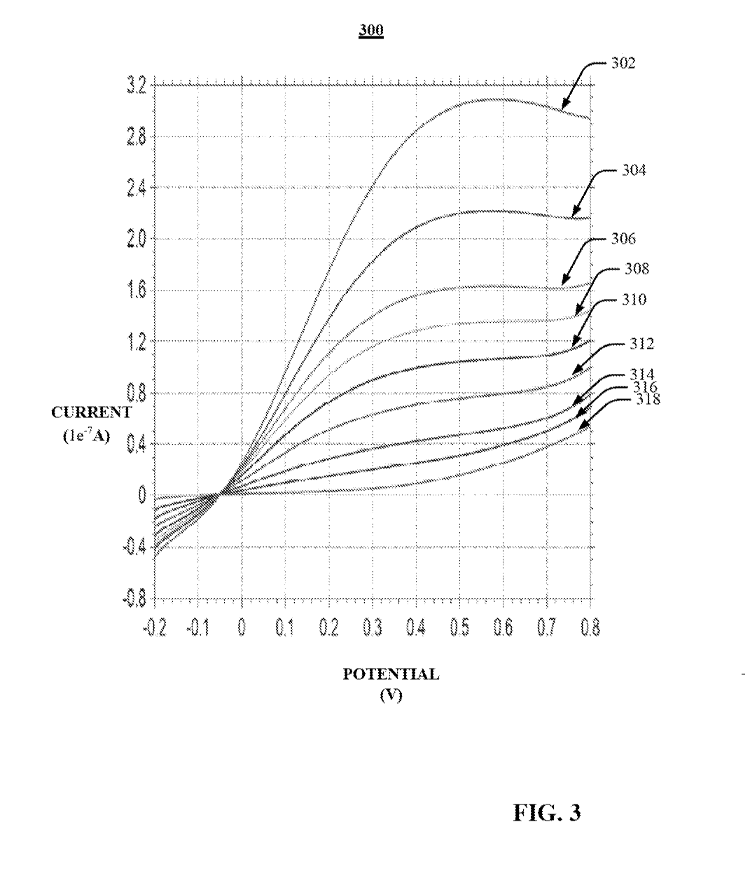

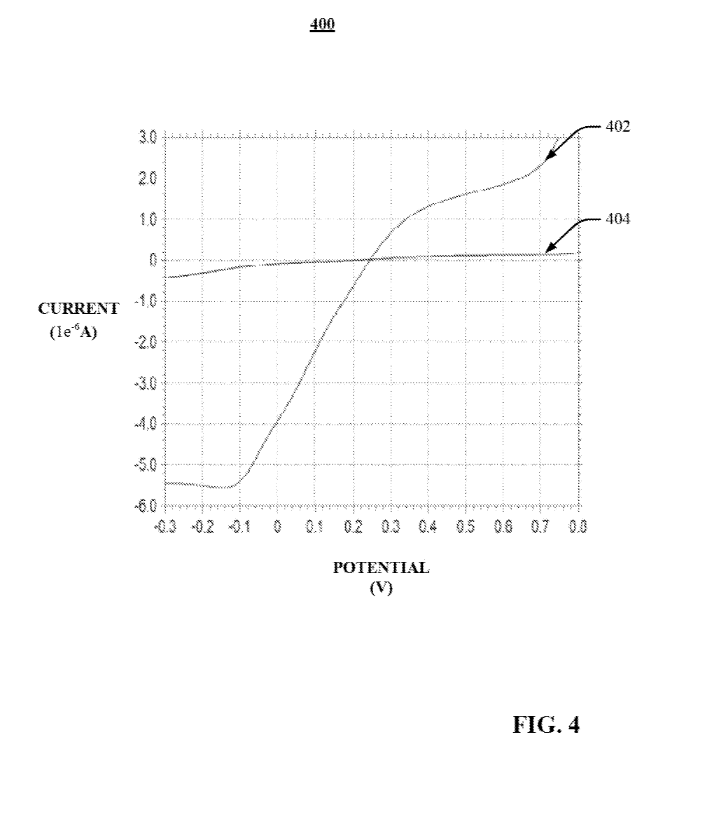

The performance of the two-electrode electrochemical sensor is illustrated with reference to FIGS. 3-7. Turning first to FIGS. 3 and 4, FIGS. 3 and 4 are illustrations of exemplary non-limiting graphs detailing the performance of two-electrode electrochemical sensors employed on a contact lens in accordance with aspects described herein. The graph of FIG. 3 illustrates linear scan voltammograms at different H₂O₂ concentrations for the 2-electrode mode electrochemical sensor.

The working electrode is approximately 25 microns (μm)×1 millimeter (mm) while the combination reference-counter electrode is Pt and approximately 0.5 mm×5 mm. The graph depicts the current (in 1e⁻⁷ Amperes (A)) as a function of the potential (volts (V)). The conditions are as follows: initial voltage=−0.2V, final voltage=0.8V, scan rate=0.05 V/second (V/s) and sample interval=0.001 V.

The concentrations of H₂O₂ per curve are as follows. Specifically, the curves show the current as the potential is varied. Curve 302 illustrates the current-potential curve for the H₂O₂ concentration of 1000 micromolars (μM); curve 304 illustrates the current-potential curve for the H₂O₂ concentration of 50 μM; curve 306 illustrates the current-potential curve for the H₂O₂ concentration of 100 μM; curve 308 illustrates the current-potential curve for the H₂O₂ concentration of 200 μM; curve 310 illustrates the current-potential curve for the H₂O₂ concentration of 300 μM; curve 312 illustrates the current-potential curve for the H₂O₂ concentration of 400 μM; curve 314 illustrates the current-potential curve for the H₂O₂ concentration of 500 μM; curve 316 illustrates the current-potential curve for the H₂O₂ concentration of 700 μM; and curve 318 illustrates the current-potential curve for the H₂O₂ concentration of 0 μM.

As shown, as the potential increases, the output current for the sensed substance substantially increases. The increase in output current is greater with greater concentration of H₂O₂.

Turning now to FIG. 4, shown are the current-potential curves for two Pt electrodes with very different sizes in a 500 micromolar H₂O₂ solution. The conditions are as follows: initial voltage=0.8V, final voltage=−0.3V, scan rate=0.01 V/second (V/s) and sample interval=0.001 V.

Curve 402 shows the performance for a Pt counter electrode having a size of approximately 0.5 mm×5 mm while curve 404 shows the performance for a Pt working electrode having a size of approximately 100 μm×1 mm. Accordingly, the Pt working electrode is much smaller than the Pt counter electrode. As shown in FIG. 4, the small oxidation current resulting from the small working electrode is balanced with the large counter electrode (i.e., which serves as the combination reference-counter electrode 206 described in this disclosure) at approximately equilibrium potential (the potential at which the current is approximately 0 A). Accordingly, FIG. 4 illustrates the electrochemical principle of this invention that enables the sensor to function as a two-electrode electrochemical sensor described with reference to FIG. 2 (e.g., electrochemical sensor 202).

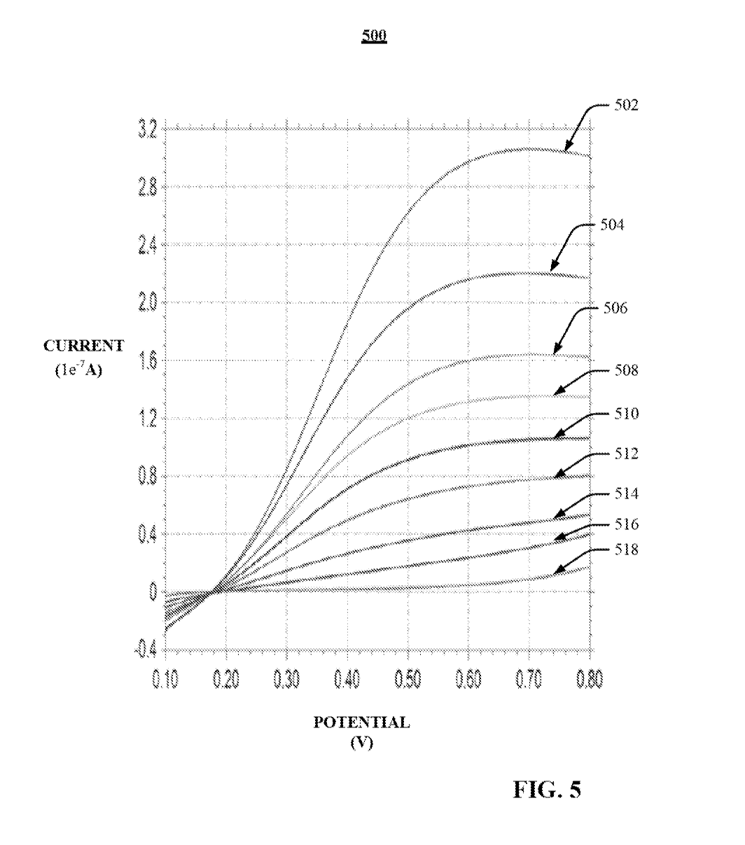

FIG. 5 is an illustration of an exemplary non-limiting graph detailing the performance of conventional three-electrode electrochemical sensors. The graphs describe a conventional three-electrode electrochemical sensor at different H₂O₂ concentrations with a Pt working electrode, a Pt counter electrode and an Ag/AgCl reference electrode. The working electrode is approximately 25 μm×1 mm while the counter electrode is Pt and approximately 0.5 mm×5 mm and the reference electrode is Ag/AgCl. The conditions are as follows: initial voltage=0.1V, final voltage=0.8V, sc^{an} rate=0.05 V/second (V/s) and sample interval=0.001 V.

The concentrations of H₂O₂ per curve are as follows. Specifically, the curves show the current as the potential is varied. Curve 502 illustrates the current-potential curve for the H₂O₂ concentration of 1000 μM; curve 504 illustrates the current-potential curve for the H₂O₂ concentration of 50 μM; curve 506 illustrates the current-potential curve for the H₂O₂ concentration of 100 μM; curve 508 illustrates the current-potential curve for the H₂O₂ concentration of 200 μM; curve 510 illustrates the current-potential curve for the H₂O₂ concentration of 300 μM; curve 512 illustrates the current-potential curve for the H₂O₂ concentration of 400 μM; curve 514 illustrates the current-potential curve for the H₂O₂ concentration of 500 μM; curve 516 illustrates the current-potential curve for the H₂O₂ concentration of 700 μM; and curve 518 illustrates the current-potential curve for the H₂O₂ concentration of 0 μM.

As shown, as the potential increases, the output current for the sensed substance substantially increases. The increase in output current is greater with greater concentration of H₂O₂.

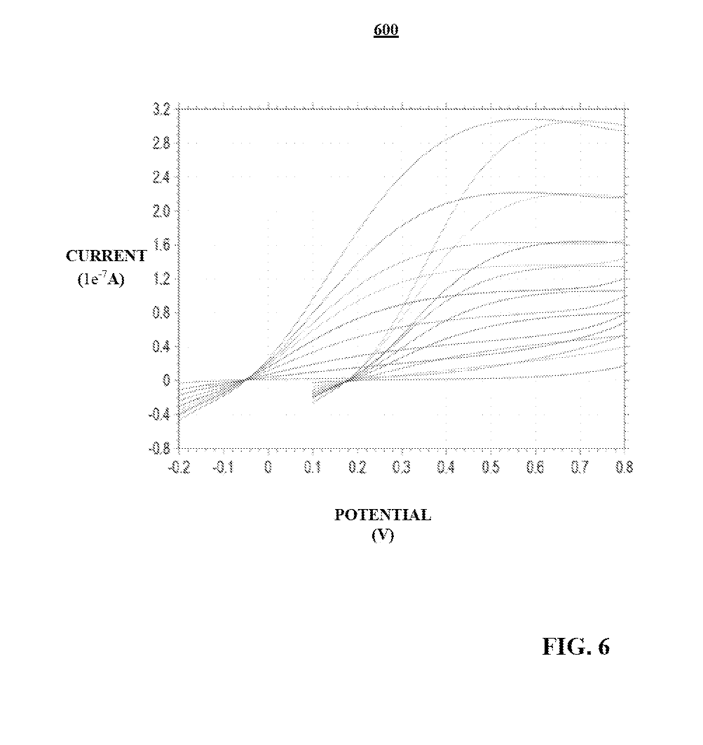

FIG. 6 is an illustration of an exemplary non-limiting graph detailing a comparison of the two-electrode electrochemical sensor employed on a contact lens in accordance with aspects described herein, and a conventional three-electrode mode electrochemical sensor.

In particular, FIG. 6 shows an overlap of all the voltammograms obtained using two-electrode and three-electrode mode configurations for electrochemical sensors. The leftmost set of curves, starting at initial voltage of −0.2V, are those associated with the two-electrode electrochemical sensor described with reference to FIG. 3 and the rightmost set of curves, starting at 0.1V, are those associated with the three-electrode electrochemical sensor described with reference to FIG. 5.

As described with reference to FIG. 3, for the two-electrode sensor, the working electrode is approximately 25 μm×1 mm while the combination reference-counter electrode is Pt and approximately 0.5 mm×5 mm. For the three-electrode mode sensor, the working electrode is approximately 25 μm×1 mm while the counter electrode is Pt and approximately 0.5 mm×5 mm and the reference electrode is Ag/AgCl.

As shown, the performance illustrated via the linear scan voltammograms using the two-electrode electrochemical sensor is equivalent to the linear scan voltammograms using the conventional three-electrode mode electrochemical sensor.

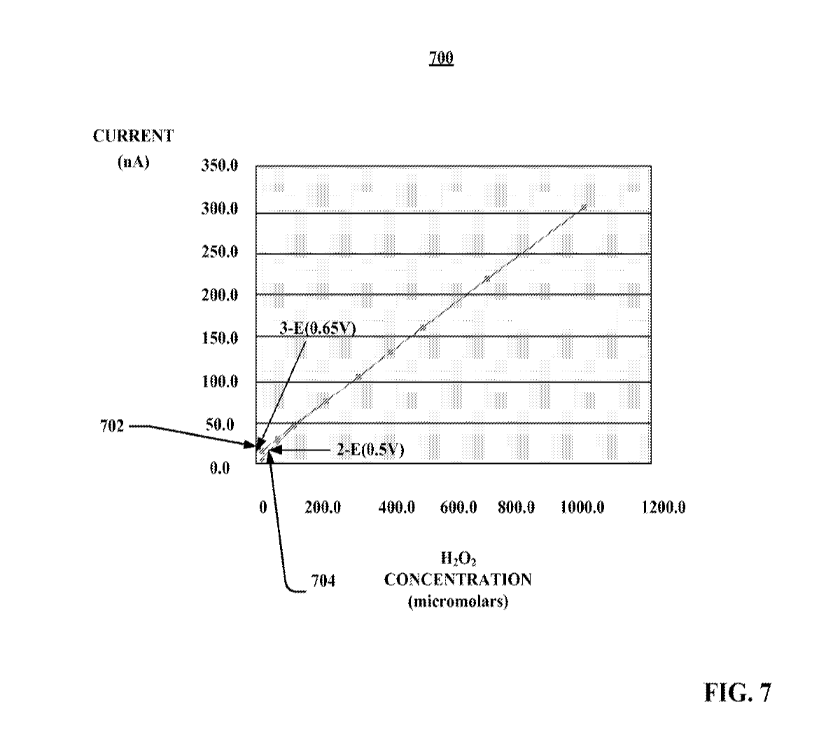

FIG. 7 is an illustration of an exemplary calibration curve for the results shown in FIG. 6. The curve 702 is associated with the two-electrode electrochemical sensor and the curve 704 is associated with the three-electrode electrochemical sensor. The current is measured in nanoamperes (nA) with the lowest value being 0 nA and the values increasing along the axis as follows: 50.0, 100.0, 150.0, 200.0, 250.0, 300.0 and 350.0. The H₂O₂ concentration is measured in μM with the lowest value being 0 and the values increasing along the axis as follows: 200, 400, 600, 800, 1000, 1200.

The calibration curves show the plateau current changes with H₂O₂ concentration. It can be seen that substantially the same responses were obtained from both the two-electrode and the three-electrode mode configurations for the electrochemical sensors.

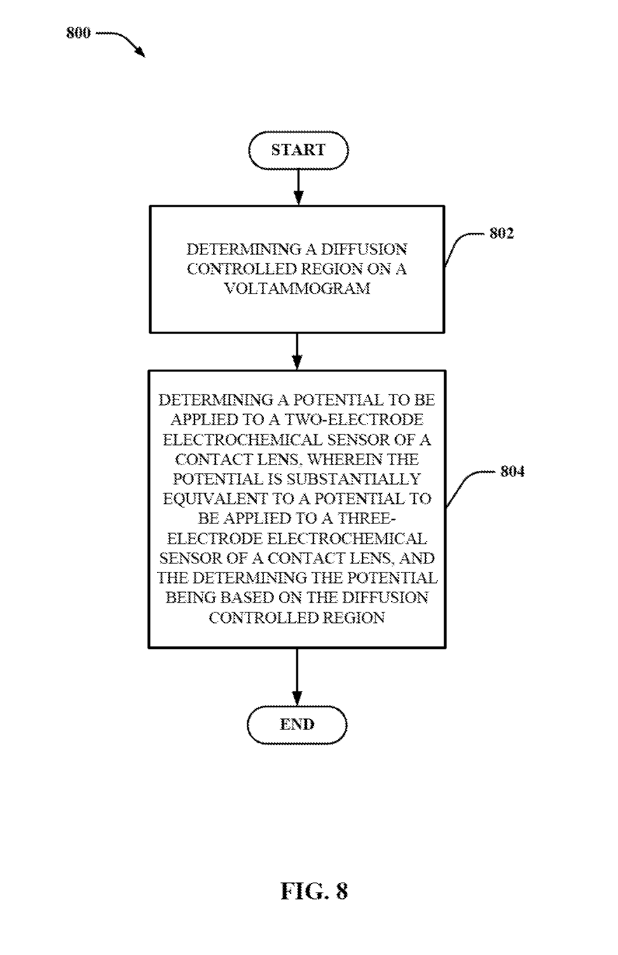

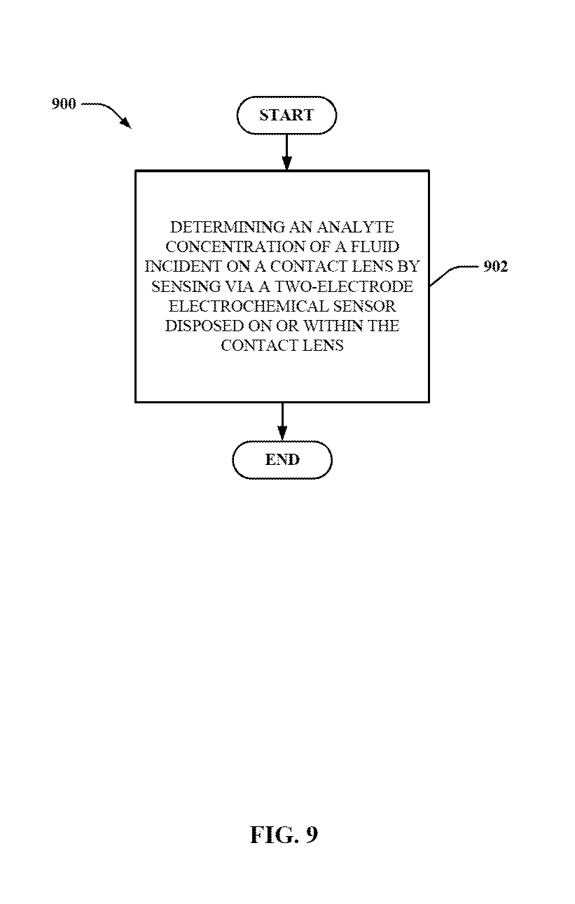

FIGS. 8 and 9 are illustrations of exemplary non-limiting flow diagrams of methods that facilitate contact lenses with two-electrode electrochemical sensors in accordance with aspects described herein.

Turning first to FIG. 8, at 802, method 800 can include determining a diffusion controlled region on a voltammogram. The diffusion controlled region can be determined based on performing one or more voltammetry experiments.

At 804, method 800 can include determining a potential to be applied to a two-electrode electrochemical sensor of a contact lens, wherein the potential is substantially equivalent to a potential to be applied to a three-electrode electrochemical sensor of a contact lens. In some aspects, determining the potential can be performed using the determined diffusion controlled region.

In some aspects, the two-electrode electrochemical sensor includes a working electrode and a combination reference-counter electrode. The working electrode and the combination reference-counter electrode can be made of a same material in some aspects, and of different material in other aspects. For example, the working electrode can be made of Pt while the combination reference-counter electrode can be made of a noble metal. As another example, the working electrode can be made of Pt while the combination reference-counter electrode can be made of C or Ag/AgCl. However, in various aspects, the ratio of the area of the combination reference-counter electrode to the area of the working electrode is between 2 and 10000.

Turning now to FIG. 9, at 902, method 900 can include determining an analyte concentration of a fluid incident on a contact lens by sensing via a two-electrode electrochemical sensor disposed on or within the contact lens (e.g., using the electrochemical sensor 202). The analyte concentration can be determined by coating the working electrode with an oxidase corresponding to the type of substance to be sensed. When the substance is brought into proximity to the two-electrochemical sensor, a chemical reaction occurs and a current is output that corresponds to the type of the substance. As such, the identity of the substance can be performed based on the output current.

### Exemplary Networked and Distributed Environments

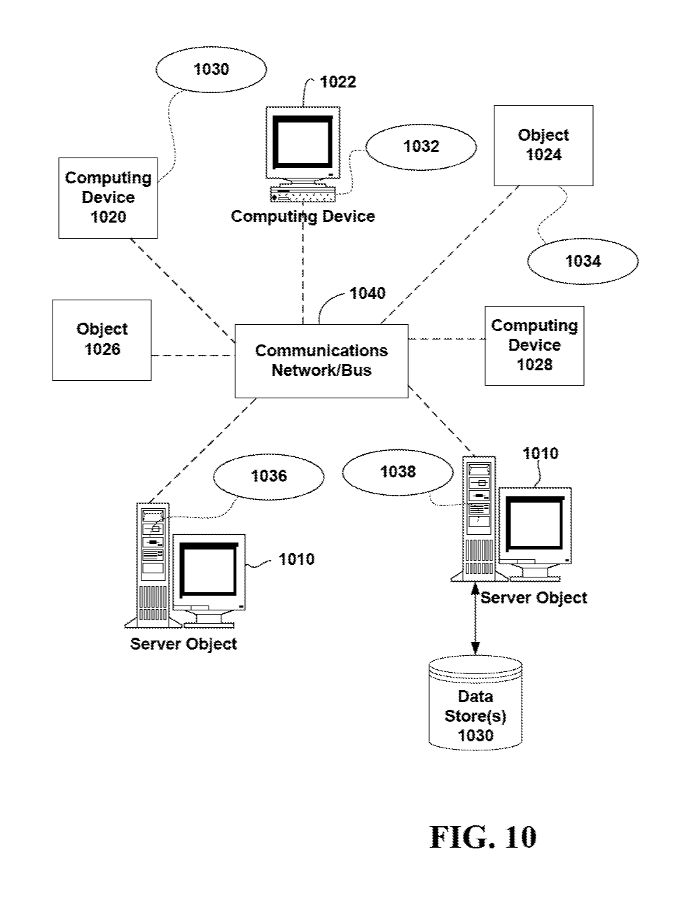

FIG. 10 provides a schematic diagram of an exemplary networked or distributed computing environment with which one or more aspects described in this disclosure can be associated. The distributed computing environment includes computing objects 1010, 1012, etc. and computing objects or devices 1020, 1022, 1024, 1026, 1028, etc., which can include programs, methods, data stores, programmable logic, etc., as represented by applications 1030, 1032, 1034, 1036, 1038. It can be appreciated that computing objects 1010, 1012, etc. and computing objects or devices 1020, 1022, 1024, 1026, 1028, etc. can include different devices, such as active contact lenses (and components thereof), personal digital assistants (PDAs), audio/video devices, mobile phones, MPEG-1 Audio Layer 3 (MP3) players, personal computers, laptops, tablets, etc.

Each computing object 1010, 1012, etc. and computing objects or devices 1020, 1022, 1024, 1026, 1028, etc. can communicate with one or more other computing objects 1010, 1012, etc. and computing objects or devices 1020, 1022, 1024, 1026, 1028, etc. by way of the communications network 1040, either directly or indirectly. Even though illustrated as a single element in FIG. 10, network 1040 can include other computing objects and computing devices that provide services to the system of FIG. 10, and/or can represent multiple interconnected networks, which are not shown.

In a network environment in which the communications network/bus 1040 can be the Internet, the computing objects 1010, 1012, etc. can be Web servers, file servers, media servers, etc. with which the client computing objects or devices 1020, 1022, 1024, 1026, 1028, etc. communicate via any of a number of known protocols, such as the hypertext transfer protocol (HTTP).

### Exemplary Computing Device

As mentioned, advantageously, the techniques described in this disclosure can be associated with any suitable device. It is to be understood, therefore, that handheld, portable and other computing devices (including active contact lens having circuitry or components that compute and/or perform various functions). As described, in some aspects, the device can be the contact lens (or components of the contact lens) and/or reader described herein. In various aspects, the data store can include or be included within, any of the memory described herein, any of the contact lenses described herein and/or the RF reader described herein. In various aspects, the data store can be any repository for storing information transmitted to or received from the contact lens.

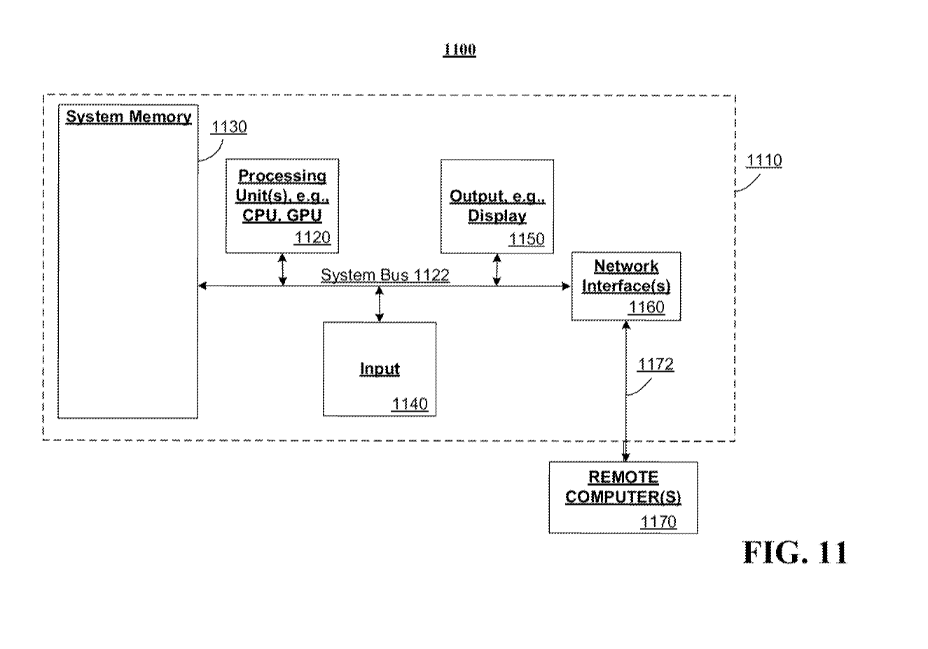

FIG. 11 illustrates an example of a suitable computing system environment 1100 in which one or aspects of the aspects described in this disclosure can be implemented. Components of computer 1110 can include, but are not limited to, a processing unit 1120, a system memory 1130, and a system bus 1122 that couples various system components including the system memory to the processing unit 1120.

Computer 1110 typically includes a variety of computer readable media and can be any available media that can be accessed by computer 1110. The system memory 1130 can include computer storage media in the form of volatile and/or nonvolatile memory such as read only memory (ROM) and/or random access memory (RAM). By way of example, and not limitation, memory 1130 can also include an operating system, application programs, other program components, and program data.

A user can enter commands and information into the computer 1110 through input devices 1140 (e.g., keyboard, keypad, a pointing device, a mouse, stylus, touchpad, touch screen, motion detector, camera, microphone or any other device that allows the user to interact with the computer 1110). A monitor or other type of display device can be also connected to the system bus 1122 via an interface, such as output interface 1150. In addition to a monitor, computers can also include other peripheral output devices such as speakers and a printer, which can be connected through output interface 1150.

The computer 1110 can operate in a networked or distributed environment using logical connections to one or more other remote computers, such as remote computer 1180. The remote computer 1180 can be a personal computer, a server, a router, a network PC, a peer device or other common network node, or any other remote media consumption or transmission device, and can include any or all of the elements described above relative to the computer 1110. The logical connections depicted in FIG. 11 include a network 1182, such local area network (LAN) or a wide area network (WAN), but can also include other networks/buses e.g., cellular networks.

Computing devices typically include a variety of media, which can include computer-readable storage media and/or communications media, in which these two terms are used herein differently from one another as follows. Computer-readable storage media can be any available storage media that can be accessed by the computer, can be typically of a non-transitory nature, and can include both volatile and nonvolatile media, removable and non-removable media. By way of example, and not limitation, computer-readable storage media can be implemented in connection with any method or technology for storage of information such as computer-readable instructions, program components, structured data, or unstructured data. Computer-readable storage media can include, but are not limited to, RAM, ROM, electrically erasable programmable read only memory (EEPROM), flash memory or other memory technology, or other tangible and/or non-transitory media which can be used to store desired information. Computer-readable storage media can be accessed by one or more local or remote computing devices, e.g., via access requests, queries or other data retrieval protocols, for a variety of operations with respect to the information stored by the medium. In various aspects, the computer-readable storage media can be, or be included within, the memory, contact lens (or components thereof) or reader described herein.

On the other hand, communications media typically embody computer-readable instructions, data structures, program components or other structured or unstructured data in a data signal such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals.

It is to be understood that the aspects described in this disclosure can be implemented in hardware, software, firmware, middleware, microcode, or any combination thereof. For a hardware aspect, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors and/or other electronic units designed to perform the functions described in this disclosure, or a combination thereof.

For a software aspect, the techniques described in this disclosure can be implemented with components or components (e.g., procedures, functions, and so on) that perform the functions described in this disclosure. The software codes can be stored in memory units and executed by processors.

What has been described above includes examples of one or more aspects. It is, of course, not possible to describe every conceivable combination of components or methodologies for purposes of describing the aforementioned aspects, but one of ordinary skill in the art can recognize that many further combinations and permutations of various aspects are possible. Accordingly, the described aspects are intended to embrace all such alterations, modifications and variations that fall within the spirit and scope of the appended claims.

Moreover, the term “or” is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form.

The aforementioned systems have been described with respect to interaction between several components. It can be appreciated that such systems and components can include those components or specified sub-components. Sub-components can also be implemented as components communicatively coupled to other components rather than included within parent components (hierarchical). Additionally, it is to be noted that one or more components can be combined into a single component providing aggregate functionality. Any components described in this disclosure can also interact with one or more other components not specifically described in this disclosure but generally known by those of skill in the art.

In view of the exemplary systems described above methodologies that can be implemented in accordance with the described subject matter will be better appreciated with reference to the flowcharts of the various figures. While for purposes of simplicity of explanation, the methodologies are shown and described as a series of blocks, it is to be understood and appreciated that the claimed subject matter is not limited by the order of the blocks, as some blocks can occur in different orders and/or concurrently with other blocks from what is depicted and described in this disclosure. Where non-sequential, or branched, flow is illustrated via flowchart, it can be appreciated that various other branches, flow paths, and orders of the blocks, can be implemented which achieve the same or a similar result. Moreover, not all illustrated blocks may be required to implement the methodologies described in this disclosure after.

In addition to the various aspects described in this disclosure, it is to be understood that other similar aspects can be used or modifications and additions can be made to the described aspect(s) for performing the same or equivalent function of the corresponding aspect(s) without deviating there from. Still further, multiple processing chips or multiple devices can share the performance of one or more functions described in this disclosure, and similarly, storage can be provided across a plurality of devices. The invention is not to be limited to any single aspect, but rather can be construed in breadth, spirit and scope in accordance with the appended claims.

1. A contact lens, comprising:
a substrate that forms at least part of a body of the contact lens; and
a circuit, disposed on or within the substrate, wherein the circuit comprises a communication component and a two-electrode electrochemical sensor, wherein the communication component comprises an antenna, and wherein the two-electrode electrochemical sensor comprises:
a working electrode; and
a combination reference-counter electrode.

2. The contact lens of claim 1, wherein the working electrode generates a signal indicative of a sensed analyte.

3. The contact lens of claim 2, wherein the combination reference-counter electrode passes the signal generated from the working electrode.

4. The contact lens of claim 1, wherein the working electrode and the combination reference-counter electrode are comprised of a same material.

5. The contact lens of claim 1, wherein the working electrode is comprised of platinum and the combination reference-counter electrode is comprised of a noble metal.

6. The contact lens of claim 1, wherein the combination reference-counter electrode is comprised of at least one of carbon or silver/silver chloride.

7. The contact lens of claim 1, wherein the two-electrode electrochemical sensor comprises a hydrogen peroxide sensor with two platinum electrodes.

8. The contact lens of claim 1, wherein the two-electrode electrochemical sensor is an amperometric sensor.

9. The contact lens of claim 8, wherein the amperometric sensor is a biosensor that senses a level of at least one of glucose, protein, triglycerides, urea or lactate in a body of a wearer of the contact lens.

10. The contact lens of claim 8, wherein the amperometric sensor comprises a working electrode having glucose oxidase deposited on the working electrode.

11. The contact lens of claim 1, wherein the working electrode is smaller than the combination reference-counter electrode.

12. The contact lens of claim 11, wherein a ratio of an area of the combination reference-counter electrode and an area of the working electrode is between 2 and 10000.

13-20. (canceled)
